(19) United States
(12) Patent Application Publication
(10) Pub. No.: US 2014/0275239 A1
(43) Pub. Date: Sep. 18, 2014

(54) Dronedarone Formulation

(71) Applicant: Mylan Inc., Canonsburg, PA (US)

(72) Inventors: Thomas D. Reynolds, Morgantown, WV (US); Boyong Li, Morgantown, WV (US)

(21) Appl. No.: 14/216,273

(22) Filed: Mar. 17, 2014

Related U.S. Application Data
(60) Provisional application No. 61/790,691, filed on Mar. 15, 2013.

(51) Int. Cl. A61K 47/38; A61K 31/343

(52) U.S. Cl. CPC A61K 47/38; A61K 31/343
USPC 514/469; 264/109

(57) ABSTRACT

Dronedarone formulations. Formulations of dronedarone are disclosed that provide consistent release and absorption of dronedarone that is independent of food ingestion. The disclosed formulation achieves this result through the inclusion of rate-controlling polymers, such as cellulose derivatives, acrylic polymers, and natural polymers. The dosage form may be prepared through blending, dry granulation, or wet granulation, followed by compression into a tablet.

### CROSS-REFERENCE TO RELATED APPLICATIONS

This application claims the benefit under 35 U.S.C. §119(e) of the earlier filing date of U.S. Provisional Patent Application No. 61/790,691 filed on Mar. 15, 2013.

### BACKGROUND OF THE INVENTION

1. Field of the Invention

The present invention relates generally to pharmaceutical formulations and methods of their manufacture. More particularly, the present invention is directed to oral dosage forms of dronedarone that provide consistent absorption of dronedarone independent of food ingestion.

2. Description of the Background

Dronedarone hydrochloride is the active drug substance in MULTAQ™, which was approved by the US FDA in 2009. Dronedarone hydrochloride is used as a treatment for atrial fibrillation.

U.S. Pat. No. 8,318,800 (which is hereby incorporated by reference) discloses that the inclusion of non-ionic surfactants in the dronedarone hydrochloride dosage form provided a consistent absorption of dronedarone that is independent of food ingestion, reducing variability in blood levels of dronedarone.

Though the desired reduction in variability is achieved using these formulations, the prior art places strict limitations on the choice of excipients available to formulators. Thus, there has been a long-standing need in the pharmaceutical community for diverse formulations of dronedarone. The present invention addresses this need.

### SUMMARY OF THE INVENTION

The present invention addresses the limitations currently existing within the art and provides dosage forms and methods of their formulation for benzofuran compounds, in particular dronedarone. The dronedarone-containing formulations achieve a release profile of dronedarone that is independent of food ingestion. Unlike the prior art, the present invention need not include non-ionic surfactants. Instead, the formulations include rate-controlling polymers to achieve the desired release profile.

It is an object of the present invention to provide a dosage form that can achieve the consistent release of dronedarone, where the dosage form includes dronedarone or a pharmaceutically acceptable salt thereof, a diluent, and a rate-controlling polymer. The formulation may optionally include binders, lubricants, glidants, coloring agents, or combinations thereof.

The rate-controlling polymer may be selected from the group consisting of cellulose derivatives, acrylic polymers, polyethylene oxide, chitosan (poly-(D-glucosamine)), gelatin, sodium alginate, pectin, scleroglucan, xanthan gum, guar gum, poly co-(methylvinyl ether/maleic anhydride), alginic acid derivatives, and combinations thereof. The rate-controlling polymer may be present at a concentration of about 1% to about 50% by weight of the dosage form.

Dronedarone may be present in the dosage forms of the present invention as dronedarone hydrochloride and may be present at a dosage of about 50 mg to about 800 mg per dosage form.

It is a further object of the present invention to provide a method for formulating such dronedarone-containing dosage forms. The methods may include the steps of first combining a diluent and a controlled-release polymer into a homogenous mixture. Dronedarone is then incorporated into that homogenous mixture, which is, in turn, compressed into a tablet. The incorporation of dronedarone may be performed as geometric progression.

The method may also include a step where, after the dronedarone is incorporated into the mixture, additional inert excipients are also mixed into the homogenous mixture. Those excipients may include lubricant, glidant, coloring agent, or combinations thereof. The initial combining step may be achieved through blending, wet granulation, or dry granulation.

### DETAILED DESCRIPTION OF THE INVENTION

It is to be understood that the figures and descriptions of the present invention have been simplified to illustrate elements that are relevant for a clear understanding of the invention, while eliminating, for purposes of clarity, other elements that may be well known. The detailed description will be provided herein below with reference to the attached drawings.

The present invention addresses the limitations currently existing within the art and provides methods and dosage forms for the formulation of dronedarone-containing dosage forms. These dosage forms do not display a “food effect” in that they provide a consistent absorption of dronedarone that is independent of food ingestion. These results are achieved without the inclusion of a non-ionic surfactant. Instead, the formulations and methods of the present invention achieve the desired release profile through inclusion of rate-controlling polymers. The rate-controlling polymers may be used in a matrix-based formulation to achieve the desired release characteristics for dronedarone. The tablets of the present invention exhibit very similar in vitro dissolution profiles as compared to the MULTAQ™ tablets.

The present specification will first provide a description of the components of the formulations of the present invention, followed by an exemplary method for formulating the dosage forms of the present invention.

The formulations of the present invention may include benzofuran compounds as active pharmaceutical ingredients. This class of compounds includes amiodarone and dronedarone. Either compound may be present as a pharmaceutically acceptable salt. One of skill in the art will recognize numerous counter-ions that may be employed appropriately in the context of the present invention. In some embodiments, dronedarone is present as dronedarone hydrochloride. Dronedarone may be present in a unit dosage form in a range from about 50 mg to about 500 mg, with 200 mg to 400 mg being particular useful within the context of the present invention.

The formulations of the present invention may include a rate-controlling polymer, which can act as a controlled-release matrix. One of skill in the art would recognize a wide variety of chemical components that could serve this role within the context of the present invention. Several examples of rate-controlling polymers useful within the context of the present invention include, but are not limited to, cellulose derivatives, acrylic polymers such as carbomer and its derivatives (e.g., POLYCARBOPHYL, CARBOPOL™), polyethylene oxide (POLYOX), chitosan (poly-(D-glucosamine)), natural polymers such as gelatin, sodium alginate, pectin, scleroglucan, xanthan gum, guar gum, poly co-(methylvinyl ether/maleic anhydride), and alginic acid derivatives.

As noted, the formulations of the present invention may include a water-swellable cellulose derivative. One of skill in the art will recognize that numerous water-swellable cellulose derivatives exist. Several examples of water-swellable cellulose derivatives that useful in the in the extended release dosage forms of the present invention, include but are not limited to, may be selected from one or more of hydroxypropyl methylcellulose (HPMC), hydroxypropylcellulose, methylcellulose, carboxy methylcellulose, hydroxy methylcellulose, and hydroxy ethylcellulose. In particular, a suitable cellulose derivative is hydroxypropyl methylcellulose. Hydroxypropyl methylcellulose is commercially available as METHOCEL™, which is manufactured by Dow Chemicals and available in various grades. A particularly useful grade of METHOCEL™ is the E15 Premium LV. The water-swellable cellulose derivative may constitute from about 1% to about 50% by weight of the total weight of formulation.

Another particularly useful rate-controlling polymer is poly(ethylene oxide), also known as POLYOX. This is a water-soluble, controlled-release resin available in numerous grades and molecular weights from Dow Chemicals.

As noted above, alginic acid derivatives may be used as rate-controlling polymers. Suitable alginic acid derivatives include, but are not limited to, alginic acid and its physiologically acceptable salts such as those of sodium, potassium, magnesium and calcium. The concentration of alginic acid derivatives may vary from about 0.1% to about 15% by weight of the total weight of formulation.

As also noted above, the rate-controlling polymers of the present invention may be cationic polymers. Suitable cationic polymers for the present invention include, but are not limited to, methacrylic acid derivatives with a dimethylaminoethyl ammonium group. In particular, the EUDRAGIT™ series of polymers marketed by Rohm Pharma may be selected. The weight of cationic polymer in the formulation may vary from about 0.1% to about 15% by weight with respect to the total weight of the formulation.

The formulations of the present invention may also include thickening agents. Suitable thickening agents within the context of the present invention include, but are not limited to, acacia gum, tragacanth gum, glyceryl behenate, glyceryl monostearate, and propylene glycol monopalmitostearate. The weight of thickening agent in the formulation may vary from about 1% to about 25% by weight with respect to the total weight of the formulation.

The formulations of the present invention may also include other pharmaceutically inert excipients such as binders, diluents, lubricants, glidants, and coloring agents.

Suitable binders may be selected from one or more of methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate, and propylene glycol.

Suitable diluents may be selected from one or more of calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol, sorbitol starch, starch pregelatinized, sucrose, sugar compressible, sugar confectioners, and mixtures thereof.

Lubricants and glidants may be selected from one or more of colloidal anhydrous silica, stearic acid, magnesium stearate, calcium stearate, talc, hydrogenated castor oil, sucrose esters of fatty acid, microcrystalline wax, yellow beeswax, and white beeswax.

Suitable colors may be selected from any FDA approved colors for internal use. Additionally, the formulation may be coated using any physiologically appropriate coating well known in the art.

The dosage forms of the present invention may be formulated in the following manner. The extended-release matrix tablet may be prepared by blending diluent and controlled-release polymers into a homogenous blend; incorporating the active drug ingredient into the blend in geometric progression; mixing with lubricant and glidant; and directly compressing into tablets. Alternatively, dry granulation or wet granulation methods may also be employed.

In one embodiment, a process for preparing extended release matrix tablets includes a wet granulation process, followed by drying the granules and compressing them into tablets.

The dosage forms of the present invention may thus be formulated by mixing components in a geometric progression. Mixing solid ingredients in a geometric progression generally refers to a process of adding almost equal amounts of two ingredients and then mixing to form a homogenous mixture of the two. This process is repeated by further mixing equal amounts to the mixture until the entire first ingredient is consumed. The entire mixture then is divided into, for example, four equal proportions and small amounts are taken from each portion and mixed thoroughly. This mixing is continued by adding from each portion until all the portions are completely used. The mixture then is further divided into two portions and the above process is repeated and ultimately the entire mixture is mixed randomly.

The following examples provide particular embodiments of the present invention. These embodiments merely provide specific details of possible implementations of the present invention and should be construed as limiting.

### EXAMPLE 1

The following components were utilized to formulate a tablet dosage form containing dronedarone hydrochloride.

| Ingredients | Mg/unit | % |
|---|---|---|
| Part I | | |
| Hypromellose, 2910 | 21.1 | 3.25 |
| Purified water, q.s. | (147.6) | |
| Ethyl alcohol, q.s. | (36.9) | |
| Part II | | |
| Dronedarone | 426.0 | 65.5 |
| Hydrochloride | | |
| Lactose | 45.8 | 7.1 |
| monohydrate | | |
| Pregelatinized | 45.6 | 7.0 |
| Starch | | |
| Polyethylene oxide | 40.0 | 6.15 |
| (WSR N80) | | |
| Crospovidone | 19.5 | 3.0 |
| Silicon Dioxide | 19.5 | 3.0 |
| Part III | | |
| Crospovidone | 13.0 | 2.0 |
| Silicon Dioxide | 13.0 | 2.0 |
| Magnesium stearate | 6.5 | 1.0 |

The Part II mixture was blended and granulated with the Part I solution, using additional portions of the water/ethanol mixture as necessary to achieve the desired consistency. The resulting granulate was then mixed with the Part III material, screened, and compressed into tablets. A clear coat can optionally be applied.

### EXAMPLE 2

The following components were utilized to formulate a tablet dosage form containing dronedarone hydrochloride.

| Ingredients | Mg/unit | % |
|---|---|---|
| Part I | | |
| Hypromellose, 2910 | 21.1 | 3.25 |
| Purified water, q.s. | (154.4) | |
| Part II | | |
| Dronedarone | 426.0 | 65.5 |
| Hydrochloride | | |
| Lactose | 45.8 | 7.1 |
| monohydrate | | |
| Pregelatinized | 45.6 | 7.0 |
| Starch | | |
| Hypromellose | 40.0 | 6.15 |
| (Methocel E15 LV) | | |
| Crospovidone | 19.5 | 3.0 |
| Silicon Dioxide | 19.5 | 3.0 |
| Part III | | |
| Crospovidone | 13.0 | 2.0 |
| Silicon Dioxide | 13.0 | 2.0 |
| Magnesium stearate | 6.5 | 1.0 |

Just with Example 1, the Part II mixture is blended and granulated with the Part I solution, using additional portions of the water/ethanol mixture as necessary to achieve the desired consistency. The resulting granulate is then mixed with the Part III material, screened, and compressed into tablets. A clear coat can optionally be applied.

Nothing in the above description is meant to limit the present invention to any specific materials, geometry, or orientation of elements. Many part/orientation substitutions are contemplated within the scope of the present invention and will be apparent to those skilled in the art. The embodiments described herein were presented by way of example only and should not be used to limit the scope of the invention.

Although the invention has been described in terms of particular embodiments in an application, one of ordinary skill in the art, in light of the teachings herein, can generate additional embodiments and modifications without departing from the spirit of, or exceeding the scope of, the claimed invention. Accordingly, it is understood that the drawings and the descriptions herein are proffered only to facilitate comprehension of the invention and should not be construed to limit the scope thereof.

What is claimed is:

1. A dosage form that provides for the consistent release of dronedarone, comprising:
dronedarone or a pharmaceutically acceptable salt thereof;
a diluent; and
a rate-controlling polymer.

2. The dosage form of claim 1, wherein said dronedarone is present as dronedarone hydrochloride

3. The dosage form of claim 2, wherein said dronedarone hydrochloride is at a dosage of about 50 mg to about 800 mg.

4. The dosage form of claim 1, wherein said rate-controlling polymer is selected from the group consisting of cellulose derivatives, acrylic polymers, polyethylene oxide, chitosan (poly-(D-glucosamine)), gelatin, sodium alginate, pectin, scleroglucan, xanthan gum, guar gum, poly co-(methylvinyl ether/maleic anhydride), alginic acid derivatives, and combinations thereof.

5. The dosage form of claim 4, wherein said rate-controlling polymer is present a concentration of about 1% to about 50% by weight of the dosage form.

6. The dosage form of claim 1, further comprising binders, lubricants, glidants, coloring agents, or combinations thereof.

7. The dosage form of claim 1, wherein said dosage form does not include a non-ionic surfactant.

8. A method for formulating a dosage form, comprising the steps of:
combining a diluent and a controlled-release polymer into a homogenous mixture;
incorporating dronedarone into said homogenous mixture; and
compressing said homogenous mixture into tablets.

9. The method of claim 8, wherein said incorporating is performed as a geometric progression.

10. The method of claim 8, further comprising, after said incorporating step, a mixing step comprising mixing a lubricant, glidant, or combinations thereof into said homogenous mixture.

11. The method of claim 8, wherein said combining step and said incorporating step are achieved through blending, wet granulation, or dry granulation.
